(19) United States
(12) Patent Application Publication
(10) Pub. No.: US 2026/0242712 A1
(43) Pub. Date: Aug. 20, 2026

(54) SYSTEM AND METHOD FOR ADJUSTING FILTER HEIGHT FOR A BIOPROCESSING SYSTEM

(71) Applicant: Global Life Sciences Solutions USA LLC, Marlborough, MA (US)

(72) Inventors: Saravanan BALAKRISHNAN, Karnataka, Bengaluru (IN); Prasad BAGIYANATHAN, Karnataka, Bengaluru (IN); Subhanjan CHATTOPADHYAY, Karnataka, Bengaluru (IN)

(73) Assignee: Global Life Sciences Solutions USA LLC, Marlborough, MA (US)

(21) Appl. No.: 19/160,697

(22) PCT Filed: Feb. 21, 2024
(86) PCT No.: PCT/US2024/016601

§ 371 Date: Aug. 28, 2025

(30) Foreign Application Priority Data
Mar. 11, 2023 (IN) 202311016356

(51) Int. Cl. C12M 1/00; C12M 3/00

(52) U.S. Cl. CPC C12M 23/50; C12M 23/14; C12M 23/46

(57) ABSTRACT

A bioreactor system for use in carrying out a biomanufacturing process is provided. The system includes a bioreactor vessel, an exhaust filter and associated filter heater, and a height adjustment mechanism connected to the exhaust filter and associated filter heater and the bioreactor vessel. The exhaust filter is fluidically connected to a single-use bioreactor bag located within the bioreactor vessel via at least one tube, and the height adjustment mechanism adjusts the height of the exhaust filter and associated filter heater relative to a top surface of a single-use bioreactor bag. By adjusting the height of the exhaust filter and associated filter heater tubing kinking is avoided, thereby ensuring that over pressurization of the single-use bioreactor bag is avoided.

### TECHNICAL FIELD

Embodiments of the invention relate generally to bioprocessing systems and methods and, more particularly, to a system and method for adjusting a filter height and associated filter heat for a bioprocessing system.

### BACKGROUND

A variety of vessels, devices, components and unit operations are known for carrying out biochemical and/or biological processes and/or manipulating liquids and other products of such processes. In order to avoid the time, expense, and difficulties associated with sterilizing the vessels used in biopharmaceutical manufacturing processes, single-use or disposable bioreactor bags and single-use mixer bags are used as such vessels. For instance, biological materials (e.g., animal and plant cells) including, for example, mammalian, plant or insect cells and microbial cultures can be processed using disposable or single-use mixers and bioreactors.

Increasingly, in the biopharmaceutical industry, single use or disposable containers are used. Such containers can be flexible or collapsible plastic bags that are supported by an outer rigid structure such as a stainless-steel shell or vessel. Use of sterilized disposable bags eliminates the time-consuming step of cleaning of the vessel and reduces the chance of contamination. The bag may be positioned within the rigid vessel and filled with the desired fluid for mixing. Depending on the fluid being processed, the system may include a number of fluid lines and different sensors, probes and ports coupled with the bag for monitoring, analytics, sampling, and fluid transfer. For example, a plurality of ports may typically be located at the front of the bag and accessible through an opening in the sidewall of the vessel, which provide connection points for sensors, probes and/or fluid sampling lines. In addition, a harvest port or drain line fitting is typically located at the bottom of the disposable bag and is configured for insertion through an opening in the bottom of the vessel, allowing for a harvest line to be connected to the bag for harvesting and draining of the bag after the bioprocess is complete.

Typically, an agitator assembly disposed within the bag is used to mix the fluid. Existing agitators are either top-driven (having a shaft that extends downwardly into the bag, on which one or more impellers are mounted) or bottom-driven (having an impeller disposed in the bottom of the bag that is driven by a magnetic drive system or motor positioned outside the bag and/or vessel). Most magnetic agitator systems include a rotating magnetic drive head outside of the bag and a rotating magnetic agitator (also referred to in this context as the “impeller”) within the bag. The movement of the magnetic drive head enables torque transfer and thus rotation of the magnetic agitator allowing the agitator to mix a fluid within the vessel. Magnetic coupling of the agitator inside the bag, to a drive system or motor external to the bag and/or bioreactor vessel, can eliminate contamination issues, allow for a completely enclosed system, and prevent leakage. Because there is no need to have a drive shaft penetrate the bioreactor vessel wall to mechanically spin the agitator, magnetically coupled systems can also eliminate the need for having seals between the drive shaft and the vessel.

During the cell culture process the bag is inflated and fluids and gases are introduced into the bag. Such gases can include air, CO₂, oxygen, and N₂. Additionally, culture media maybe periodically (or continuously) added throughout the culturing process. The gases and fluid are also removed from the bag during the culturing process. Typically, gasses are removed through an exhaust line (e.g., tube) that is connected to an exhaust filter attached to the outer vessel at the top of the bag. A filter heater is typically wrapped around the filter to ensure that condensation within the exhaust line is minimized, thereby reducing fouling of the exhaust filter. However, because the bag is in an inflated state and gases are being added to the system, the amount of inflation varies over the course of the cell culturing process. In a situation where the bag overinflates, the exhaust line is pushed up. Since the exhaust filter is attached to the outer vessel, it has a fixed height. This in turn causes the exhaust line to bend or otherwise kink, causing the bag pressure to rise above safe levels (i.e., because the exhaust line is at least partially blocked, preventing gases from exiting the bag at a sufficient rate). Currently, when this happens the culture has to be stopped or paused, otherwise the bag would rupture due to over pressurization. A new (or the same) exhaust filter may then be attached to the bag with a shorter exhaust line and the culturing process can then be resumed. However, these steps are cumbersome, require the culturing to be paused, which can jeopardize cell viability, and necessitate additional supplies (e.g., additional tubing, connectors, etc.).

In view of the above, there is a need for a mechanism to adjust the height of the exhaust filter and associated filter heater such that overinflation of the bag does not cause the exhaust line to kink.

### BRIEF DESCRIPTION

A first aspect of the invention relates to a method for adjusting a height of an exhaust filter and associated filter heater. The method comprises: attaching the exhaust filter and associated filter heater to a height adjustment mechanism; attaching the height adjustment mechanism to a bioreactor vessel; fluidically connecting the exhaust filter to a single-use bioreactor bag located within the bioreactor vessel via at least one tube; and adjusting the height of the exhaust filter and associated filter heater relative to a top surface of the single-use bioreactor bag. According to embodiments, the step of adjusting occurs after inflating the single-use bioreactor bag so that a distance between the exhaust filter and associated filter heater relative to the top surface of the single-use bioreactor bag is increased, such that the at least one tube is straightened. By straightening the tube overpressure generated in the single-use bioreactor bag due to kinking of the at least one tube is alleviated.

In embodiments, the height adjustment mechanism comprises at least one rail, and adjusting comprises sliding the exhaust filter and associated filter heater along the rail. The height adjustment mechanism further comprises, at least one clamp; and at least one plunger, such that sliding the exhaust filter and associated filter heater along the rail comprises: rotating the plunger in a first direction such that the plunger unlocks from the rail, and moving the exhaust filter and associated filter heater along the rail. After the exhaust filter and associated filter heater are moved along the rail, the method further comprises rotating the plunger in a second direction to lock the plunger within a slot of the rail.

In further embodiments, the height adjustment mechanism comprises: a lead screw located within a channel of the at least one rail; and a knob located on a first end of the lead screw, wherein sliding the exhaust filter and associated filter heater along the rail comprises rotating the knob.

According to any embodiment, the height of the exhaust filter and associated filter heater relative to the top surface of the single-use bioreactor bag is automatically adjusted based upon a height of the top surface the single-use bioreactor bag or a sensed pressure within the single-use bioreactor bag. In one embodiment, automatically adjusting comprises activating a motor when the sensed pressure crosses a threshold.

A second aspect of the invention relates to a bioreactor system, comprising: a bioreactor vessel; an exhaust filter and associated filter heater; and a height adjustment mechanism connected to the exhaust filter and associated filter heater and the bioreactor vessel; wherein the exhaust filter is fluidically connected to a single-use bioreactor bag located within the bioreactor vessel via at least one tube; wherein the height adjustment mechanism adjusts the height of the exhaust filter and associated filter heater relative to a top surface of a single-use bioreactor bag.

In embodiments, the height adjustment mechanism comprises: at least one rail, wherein the height of the exhaust filter and associated filter heater are adjusted by sliding along the at least one rail. The height adjustment mechanism further comprises: at least one clamp; and at least one plunger, wherein rotation of the plunger in a first direction unlocks the plunger from the rail, such that the exhaust filter and associated filter heater can travel along the rail. Rotation of the plunger in a second direction locks the plunger within a slot the rail, such that the exhaust filter and associated filter heater cannot move along the rail.

In further embodiments, the height adjustment mechanism further comprises: a lead screw located within a channel of the at least one rail; and a knob located on a first end of the lead screw, wherein the height of the exhaust filter and associated filter heater are adjusted by rotating the knob.

According to any embodiment, the system further comprises: at least one sensor configured to determine the height of the top surface or a sensed pressure within the single-use bioreactor bag; and a motor, wherein the height of the exhaust filter and associated filter heater is automatically adjusted by activation of the motor when the sensed pressure or the height of the top surface crosses a threshold.

### DRAWINGS

The present invention will be better understood from reading the following description of non-limiting embodiments, with reference to the attached drawings, wherein below:

FIG. 1 is a perspective view of a bioprocessing system according to an embodiment of the invention.

FIG. 2 is a perspective view of a component management apparatus of the bioprocessing system of FIG. 1, according to an embodiment of the present invention.

FIGS. 3A and 3B illustrate a height adjustment mechanism in an assembled and exploded view, respectively, according to an embodiment of the present invention.

FIGS. 4A-4D illustrate operation of the height adjustment mechanism of FIGS. 3A-3B to adjust the height of an exhaust filter and associated filter heater, according to an embodiment of the present invention.

FIG. 5 illustrates an alternative height adjustment mechanism, according to an embodiment of the present invention.

FIGS. 6A and 6B illustrate how operation of the height adjustment mechanism straightens a kinked tube connected to the exhaust filter, according to embodiments of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, a rigid container, or a flexible or semi-rigid tubing, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems, e.g., chromatography and tangential flow filter systems, and their associated flow paths. As used herein, the term “bag” means a flexible or semi-rigid container or vessel used, for example, as a bioreactor or mixer for the contents within. As used herein, “consumable” or “consumable component” means devices or components that are intended to be replaced regularly due to wear or use.

Embodiments of the invention provide bioprocessing systems and, in particular, exhaust filter height adjustment mechanisms for a bioreactor system. In an embodiment, a bioprocessing system includes a vessel defining an interior space for receiving a flexible bioprocessing bag, the vessel having an access door in a sidewall of the vessel and providing access to the interior space, and a component management apparatus mounted to the sidewall of the vessel and having a mounting frame for mounting of at least one consumable component of the bioprocessing system.

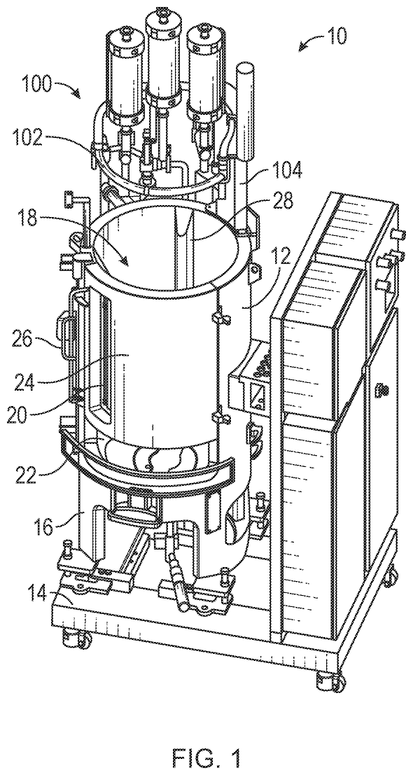

With reference to FIG. 1, a bioprocessing system 10 (also referred to herein as bioreactor system 10) according to an embodiment of the invention is illustrated. The bioreactor system 10 includes a generally rigid bioreactor vessel or support structure 12 mounted atop a base 14 having a plurality of legs 16. The vessel 12 may be formed, for example, from stainless steel, polymers, composites, glass, or other metals, and may be cylindrical in shape, although other shapes may also be utilized without departing from the broader aspects of the invention. The vessel 12 can be any shape or size as long as it is capable of supporting a single-use, flexible bioreactor bag in an interior space 18 thereof. For example, according to one embodiment of the invention the vessel 12 is capable of accepting and supporting a 10L-2000L flexible or collapsible bioprocess bag.

The vessel 12 may include one or more sight windows 20, which allows an operator to view a fluid level within the flexible bag positioned within the interior space 18, as well as a window 22 positioned at a lower area of the vessel 12. The window 22 allows access to the interior of the vessel 12 for insertion and positioning of various sensors and probes (not shown) within the flexible bag, and for connecting one or more fluid lines to the flexible bag for fluids, gases, and the like, to be added or withdrawn from the flexible bag. Sensors/probes and controls for monitoring and controlling important process parameters include any one or more, and combinations of: temperature, pressure, pH, dissolved oxygen (DO), dissolved carbon dioxide (pCO₂), mixing rate, and gas flow rate, for example.

In an embodiment, the vessel 12 includes an access door 24 hingedly or pivotally connected to a sidewall of the vessel 12 permitting access to the interior space 18. The door 24 may include a handle 26 that facilitates movement of the door between the open and closed positions. In an embodiment, the door 24 may be configured and positioned such that when the door 24 is in the closed position, a lower edge of the door 24 forms an upper edge or boundary of the window 22, and/or a side edge of the door 24 forms an edge or boundary of the window 20. By having the edges of the door 24 define one or more boundaries of the windows 22, 24, when the door 22 is in the open position, a contiguous and unobstructed access opening in the sidewall of the vessel is formed by the opening 20, opening 22 and open door 24 (i.e., the opening in which the door is received). Accordingly, the area of the contiguous access opening formed in the sidewall of the vessel 12 when the door is in the open position is equivalent to the combined areas of the door 24, window 22 and window 24. This provides greater clearance and access to the interior space 18 than would otherwise be possible if the door and windows were separated by a portion of the sidewall of the vessel 12.

With further reference to FIG. 1, the interior sidewall of the vessel 12 may include one or more vertical baffles 28 that project into the interior space 18. The baffles 28 may be generally triangular in cross-section, although shapes and configurations known in the art may also be utilized without departing from the broader aspects of the invention. The baffles 28 are configured to contact and bias the flexible bag (when installed in the interior space 18) inwardly during a bioprocessing operation, for purposes known in the art.

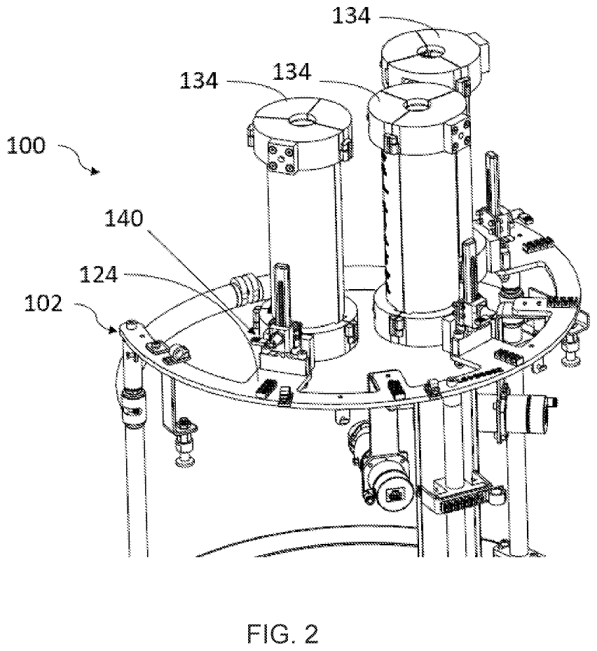

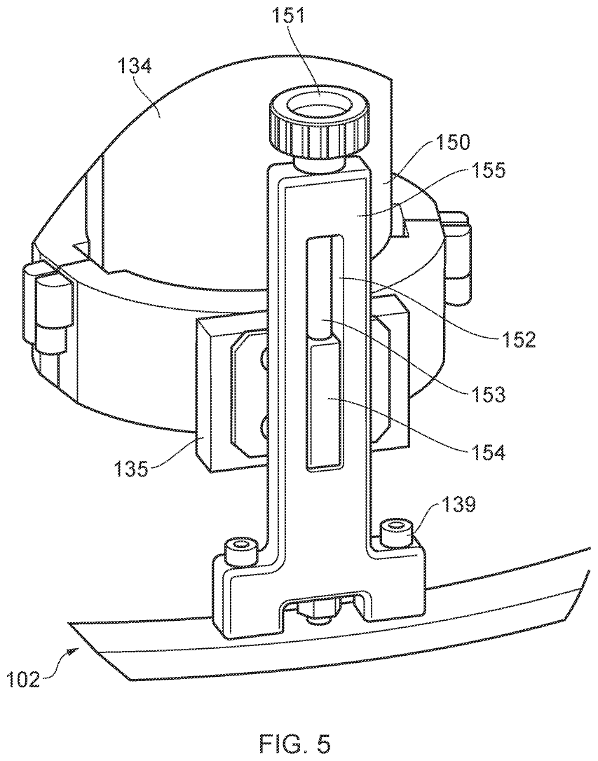

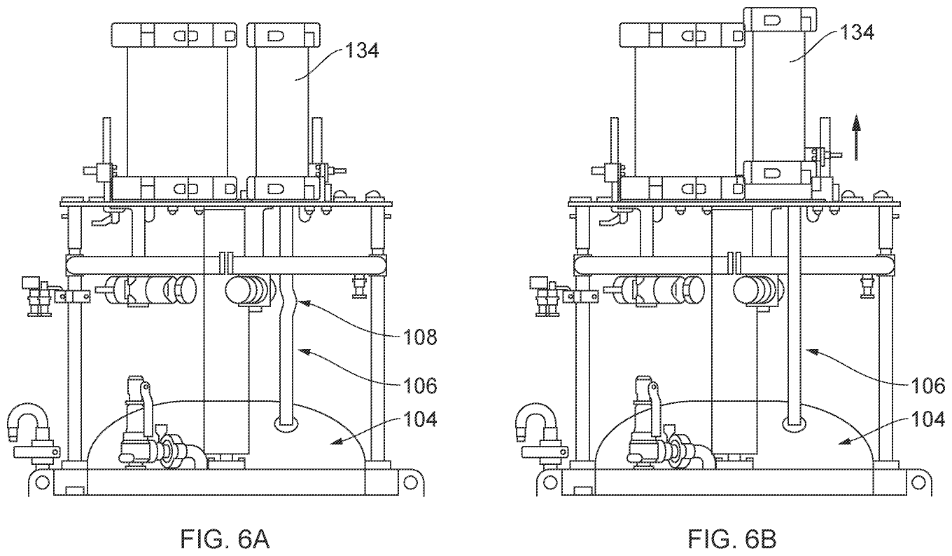

As further shown in FIGS. 1 and 2, the bioreactor system 10 also includes a component management apparatus 100. The apparatus 100 includes a frame 102, which is utilized to connect various components to the flexible bag. The frame 102 can take the form of a semi-circular or curved rail that generally mimics the outer diameter of the vessel 12. Specifically, FIG. 2 illustrates how exhaust filters with associated filter heaters 134 may be attached to frame 102. As shown, each exhaust filter and associated filter heater 134 can be attached to a frame portion 124 via a height adjustment mechanism 140. Specifically, as best shown in FIGS. 2, 3 and 5, each exhaust filter and associated filter heater 134 is attached to a height adjustment mechanism 140, 150, via fasteners 146, which in turn are attached to frame 102 via fasteners 139. As these figures show, height adjustment mechanism 140, 150 can be directly attached to the rail (FIG. 5) or a protruding portion (e.g., flange) 124 of the frame 102.

As will be discussed in greater detail below, height adjustment mechanism 140, 150 is configured to adjust the height of exhaust filter and associated filter heater 134 relative to the frame 102 and vessel 12, such that the distance between the exhaust filter and associated filter heater 134 and the single-use bag can be adjusted before, during and/or after a cell culture process is taking place within the single-use bag. Specifically, during a cell culturing process the exhaust filter and associated filter heater 134 is fluidically connected to the single-use bag, via at least one tube, such that gases introduced (and generated) during cell culturing can exit the single-use bag. The height of the exhaust filter and associated filter heater 134 can change to accommodate for changes in the inflation of the single-use bag due to changes in the amount of gases entering (or being generated in) the single-se bag. This ensures that the integrity of the fluid connection single-use bag and exhaust filter is properly maintained.

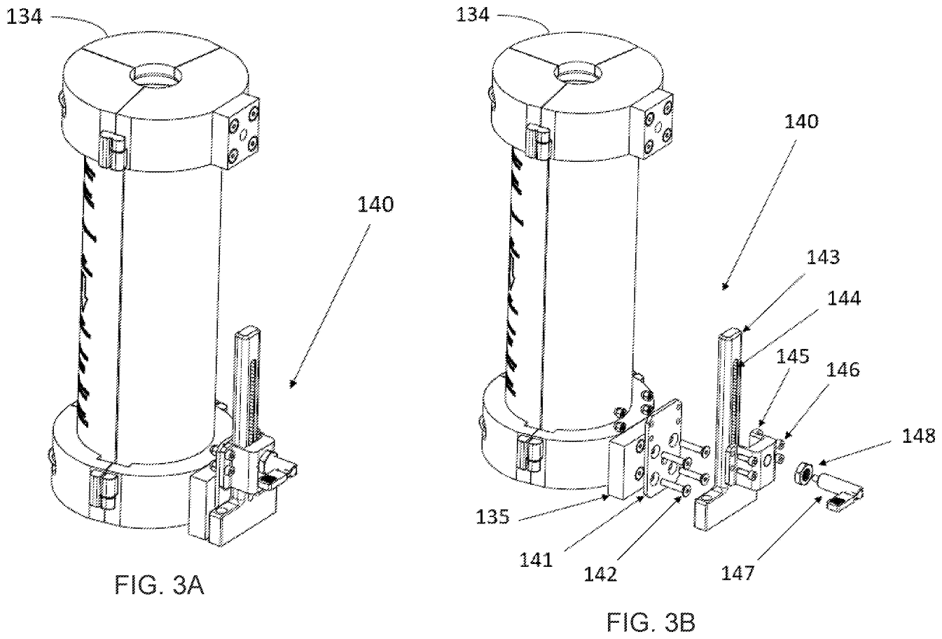

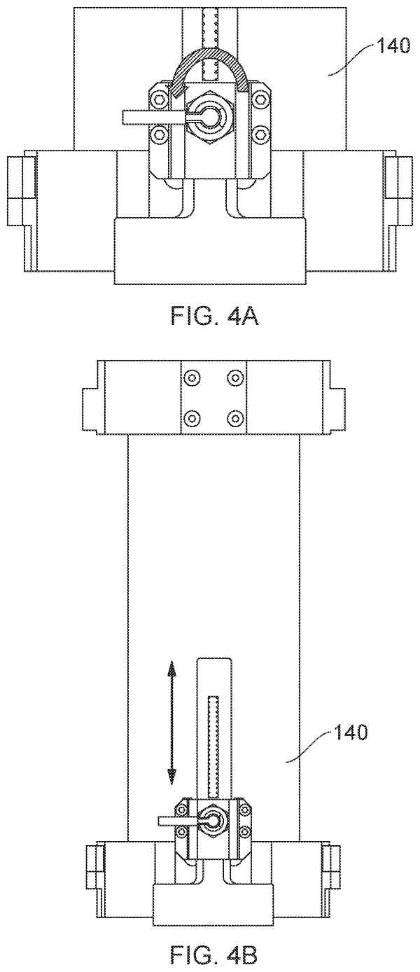

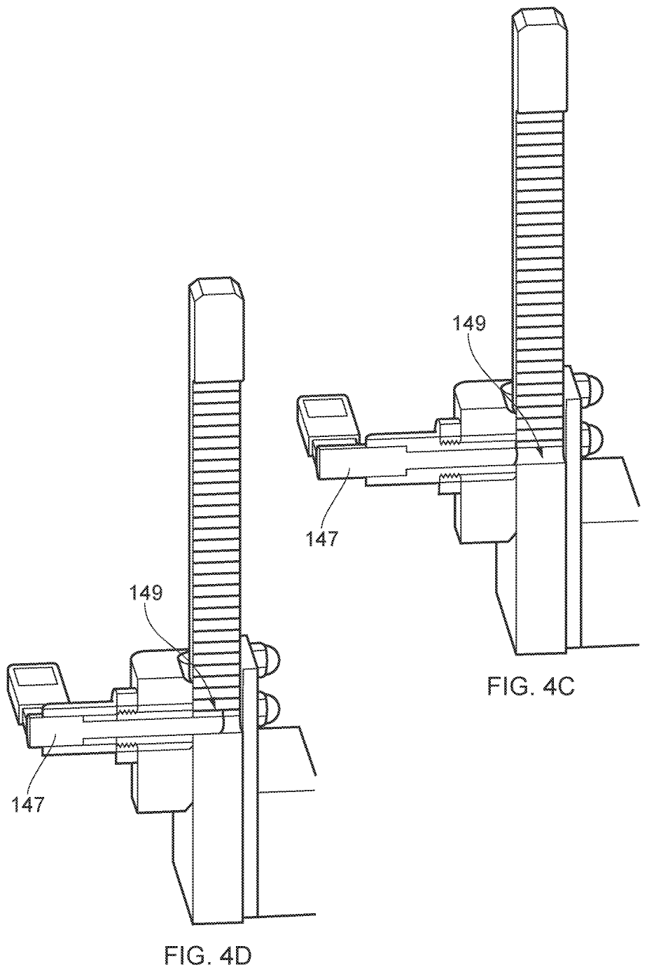

According to an embodiment of the invention, height adjustment mechanism 140 includes a back place 141 that is attached to an attachment plate 135 of the exhaust filter and associated filter heater 134 via fasteners 142. The height adjustment mechanism 140 further includes a rail 143 that is attached to the frame 102 via fasteners 139 and a clamp 146 that is attached to the back plate 141 via fasteners 146. As best illustrated in FIGS. 3A-4B, rail 143 generally has an upside down “T” shape, with a slot 144 located within the vertical section thereof. The clamp 145 is shaped such that the vertical section of rail 143 can slide within a recess of the clamp 145. Height adjustment mechanism 140 further includes a plunger 147 that is inserted into a hole in the clamp 145. The length of the plunger 147 is such that is penetrates through the clamp 145 and into the slot 144. Nut 148 is threaded onto the plunger 148. The slot 144 includes an array of protrusions (e.g., is toothed) 149 (as best seen in FIGS. 4C and 4D) such that the plunger 147 can sit within a depression (i.e., circular cutout) of the rail 143. Thus, when a handle of the plunger 147 is rotated in a first direction (e.g., clockwise) the plunger 147 is advanced into the slot 144 and sits within one of the depressions (see, e.g., FIG. 4D), thereby locking the exhaust filter and associated filter heater 134 and preventing motion of the exhaust filter and associated filter heater 134 relative to the rail 143. Similarly, when the handle of the plunger is rotated in a second direction (e.g., counterclockwise) the plunger 147 is retracted out of the depression and rail 143 (See, e.g., FIG. 4C), thereby unlocking the exhaust filter and associated filter heater 134 and allowing motion of the exhaust filter and associated filter heater 134 relative to the rail 143.

With such a configuration of components, the height adjustment mechanism 140 is able to adjust the height of the exhaust filter and associated filter heater 134. Specifically, the exhaust filter and associated filter heater 134 is slidable along the length of rail 143. In order to accomplish this, and as illustrated by FIGS. 4A-4D, a user turns the handle of plunger 147 counterclockwise (as indicated by the arrow in FIG. 4A). This unlocks the height adjustment mechanism 140. Then a user can slide the exhaust filter and associated filter heater 134 along the rail 143 to a desired height (as indicated by the arrow in FIG. 4B). To prevent further movement of the exhaust filter and associated filter heater 134, a user then turns the handle clockwise, which causes the plunger to sit within a desired depression in the rail 143, thereby locking the height of the exhaust filter and associated filter heater 134.

FIG. 5 illustrates an alternative embodiment of a height adjustment mechanism 150. As illustrated, height adjustment mechanism 150 also includes a rail 155 that has a generally upside down “T” shape. The lower portion of rail 155 is fastened to the frame 102 via fasteners 139, as previously discussed. Rail 155 has a channel 152 formed along at least a part of its length, with a leadscrew 153 spanning the length of the channel 152. The top of the leadscrew terminates at a knob 151 located on the top of the rail 155. A backplate 154, which is fastened to attachment plate 135 (and thereby attached to the exhaust filter and associated filter heater 134), has a portion thereof that protrudes into the channel in which the leadscrew 153 is threaded therethrough. Specifically, a portion of backplate 154 is configured to fit within the channel 153 and includes a bore therethrough that the leadscrew is threaded through. In this way, a portion of backplate 154 acts as a nut that is threaded onto leadscrew 153. To adjust the height of the exhaust filter and associated filter heater 134, a user rotates the knob 151. Specifically, a user rotates the knob 151 in a first direction (e.g., clockwise), causing the leadscrew 153 to also rotate in the first direction, which in turn causes backplate 154 to move up along the channel due to its threaded engagement with the leadscrew 153. Similarly, by rotating the knob 151 in a second direction (e.g., counterclockwise) the backplate 154 moves down along the channel. In this way a user can adjust the height of the exhaust filter and associated filter heater 134 by rotation of knob 151.

While the above embodiments illustrate height adjustment mechanisms having components of a specific geometry/design, the invention is not so limited. For example, the T-shaped rail can have other configurations (e.g., have a generally plate-like shape, rod shape, etc.), so long as the rail is attachable to the frame of the outer vessel. Additionally, the length of the rail can be varied based upon the particular application (i.e., length of height adjustment needed). According to one embodiment, the length of the rail 143, 152 is approximately 50-200mm, and in one preferred embodiment, the length is approximately 80 mm.

While two embodiments of height adjustment mechanisms 140, 150 have been described above, the invention is not so limited and other variations are within the scope of the invention. For example, the height adjustment mechanism may include an array of hooks, ledges, or protrusions located at various heights of the rail, while the attachment plate 135 includes a corresponding hook or attachment point such that the attachment plate 135 can be hung or otherwise attached to the rail at various heights. Still further, backplate 135 and the rail can each be made from or otherwise include at least one magnet or ferromagnetic material such that the two are magnetically attracted to each other. In this way, the back plate and rail can be magnetically coupled at various heights along the rail.

According to any embodiments of the invention, the height adjustment of the exhaust filter and associated filter heater 134 can be automated. For example, a motor can be attached to knob 151 (or mounted directed to the lead screw 153) and utilized to rotate the knob 151 in the desired direction to move the exhaust filter and associated filter heater 134 up and down along rail 155. Additionally, activation of the motor can be automated according to a feedback loop. For example, and as discussed above, when the tubing attached to the exhaust filter kinks pressure within the single-use bag builds up. This increase in pressure can be measured by a pressure sensor within the single-use bag, and when the pressure crosses a threshold a signal can be generated, causing activation of the motor, which in turn rotates the knob 151 (or the leadscrew 153) and raises the exhaust filter and associated filter heater 134. Additionally, the height of the top surface of the single-use bag can be monitored, for example by way of an optical sensor, camera, or the like, and when the top surface increases in height by a predetermined amount a signal can be generated that causes activation of the motor to raise the exhaust filter and associated filter heater 134.

FIGS. 5A and 5B illustrates operation of the height adjustment mechanism 140, 150 in order to straighten at least one tube connected to an exhaust filter. As discussed above, when cell culturing is taking place within the single-use bag 104, gases and fluids are added to the bag to promote the culture process. At various times during the process, the single-use bag 104 may overinflate. When such an overinflation occurs, the distance between the top surface of the single-use bag 104 and the bottom of the exhaust filter reduces, which causes the tube 106 that fluidically connects the bag to the filter to bend. If the overinflation is of a sufficient amount, the tube 106 will bend to a degree that a kink or blockage 108 is created in the tube 106 (see, e.g., FIG. 5A). This causes pressure to build up within the single-use bag 104, jeopardizing the integrity of the bag. The present invention advantageously allows a user to unkink or otherwise prevent the blockage by raising the height of the exhaust filter and associated filter heater (see, e.g., FIG. 5B). Said another way, by raising the exhaust filter and associated filter heater 134 (see the arrow illustrated in FIG. 5B), via the presently disclosed height adjustment mechanism, the tube connecting the single-use bag to the exhaust filter is straightened, thereby ensuring that gases flow out of the bioreactor system 10 at proper rates and preventing over pressurization of the system.

It is noted that the above description describes height adjustment mechanisms to adjust the height of an exhaust filter and associated filter heater. However, the invention is not limited to the adjustment of such components, and the presently described height adjustment mechanisms can be implemented to adjust the height of any component that requires adjustment on the bioreactor system.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. A method for adjusting a height of an exhaust filter and associated filter heater, comprising:
attaching the exhaust filter and associated filter heater to a height adjustment mechanism; attaching the height adjustment mechanism to a bioreactor vessel;
fluidically connecting the exhaust filter to a single-use bioreactor bag located within the bioreactor vessel via at least one tube; and
adjusting the height of the exhaust filter and associated filter heater relative to a top surface of the single-use bioreactor bag.

2. The method of claim 1, further comprising:
inflating the single-use bioreactor bag, wherein the adjusting occurs after inflating the single-use bioreactor bag.

3. The method of claim 2, wherein a distance between the exhaust filter and associated filter heater relative to the top surface of the single-use bioreactor bag is increased such that the at least one tube is straightened, thereby relieving overpressure generated in the single-use bioreactor bag due to kinking of the at least one tube.

4. The method of any claim 1, wherein the height adjustment mechanism comprises:
at least one rail,
wherein the adjusting comprises sliding the exhaust filter and associated filter heater along the rail.

5. The method of claim 4, wherein the height adjustment mechanism further comprises:
at least one clamp; and
at least one plunger, wherein sliding the exhaust filter and associated filter heater along the rail comprises:
rotating the plunger in a first direction such that the plunger unlocks from the rail, and
moving the exhaust filter and associated filter heater along the rail.

6. The method of claim 5, further comprising:
rotating the plunger in a second direction to lock the plunger within a slot of the rail after the exhaust filter and associated filter heater have moved along the rail.

7. The method of claim 4, wherein the height adjustment mechanism further comprises:
a lead screw located within a channel of the at least one rail; and
a knob located on a first end of the lead screw, wherein sliding the exhaust filter and associated filter heater along the rail comprises rotating the knob.

8. The method of claim 1, wherein the height of the exhaust filter and associated filter heater relative to the top surface of the single-use bioreactor bag is automatically adjusted based upon a height of the top surface of the single-use bioreactor bag or a sensed pressure within the single-use bioreactor bag.

9. The method of claim 8, wherein automatically adjusting comprises activating a motor when the sensed pressure crosses a threshold.

10. A bioreactor system, comprising:
a bioreactor vessel;
an exhaust filter and associated filter heater; and
a height adjustment mechanism connected to the exhaust filter and associated filter heater and the bioreactor vessel;
wherein the exhaust filter is fluidically connected to a single-use bioreactor bag located within the bioreactor vessel via at least one tube, and
wherein the height adjustment mechanism adjusts the height of the exhaust filter and associated filter heater relative to a top surface of a single-use bioreactor bag.

11. The system of claim 10, wherein the height adjustment mechanism comprises:
at least one rail,
wherein the height of the exhaust filter and associated filter heater are adjusted by sliding along the at least one rail.

12. The system of claim 11, wherein the height adjustment mechanism further comprises:
at least one clamp; and
at least one plunger,
wherein rotation of the plunger in a first direction unlocks the plunger from the rail, such that the exhaust filter and associated filter heater can travel along the rail.

13. The system of claim 12, wherein rotation of the plunger in a second direction locks the plunger within a slot of the rail, such that the exhaust filter and associated filter heater cannot move along the rail.

14. The system of claim 10, wherein the height adjustment mechanism further comprises:
a lead screw located within a channel of the at least one rail; and
a knob located on a first end of the lead screw, wherein the height of the exhaust filter and associated filter heater are adjusted by rotating the knob.

15. The system of claim 14, further comprising:
at least one sensor configured to determine the height of the top surface or a sensed pressure within the single-use bioreactor bag; and
a motor,
wherein the height of the exhaust filter and associated filter heater is automatically adjusted by activation of the motor when the sensed pressure or the height of the top surface crosses a threshold.
